(19) United States
(12) Patent Application Publication
(10) Pub. No.: US 2026/0051401 A1
(43) Pub. Date: Feb. 19, 2026

(54) EARLY STAGE DETECTION OF PERITONITIS

(71) Applicant: Fresenius Medical Care Holdings, Inc., Waltham, MA (US)

(72) Inventors: Louis LeeGrande Barrett, West Point, UT (US); Kristian Sammann, Murray, UT (US); Ronald Scott Glaittli, Bountiful, UT (US); David Wayne Peterson, Clinton, UT (US); Eric Bergman, Newton, MA (US)

(21) Appl. No.: 18/805,178

(22) Filed: Aug. 14, 2024

(51) Int. Cl. G16H 50/20; G01N 21/53; G01N 33/49

(52) U.S. Cl. CPC G16H 50/20; G01N 21/532; G01N 33/49; G01N 2800/347

(57) ABSTRACT

The present teachings include optical systems and methods for detection of peritonitis in early stages for patients undergoing a peritoneal dialysis (PD) treatment. An initial measurement of optical extinction of effluent fluid may be stored as a baseline value for a patient, e.g., to compensate for color, density, and similar optical factors related to the specific patient's physiology. Optical extinction through the effluent may be periodically measured over the course of a PD treatment, and extinction of signal relative to the baseline is analyzed, primarily for early indicators of potential peritonitis. The system may be externally connected to an effluent conduit, e.g., via a clip. A technique of ratioing an optical signal through effluent to a reference optical signal for measurements may provide high sensitivity while filtering out significant noise, enabling early detection of infection. Alerts of potential infection may be provided to the patient, caregiver, and/or medical team.

### FIELD

The present disclosure generally relates to devices, systems, and methods for detecting peritonitis in early stages in patients undergoing a peritoneal dialysis (PD) treatment.

### BACKGROUND

Peritoneal dialysis (PD) is a renal replacement therapy for patients who suffer from renal disease. Techniques for peritoneal dialysis include Continuous Ambulatory Peritoneal Dialysis (CAPD) and Automated Peritoneal Dialysis (APD). CAPD is performed on an ongoing basis, where spent dialysate drains into a drain bag that is later discarded and replaced. APD uses a cycler to deliver and drain the dialysate, typically while the patient is stationary (e.g., while sleeping). In APD, spent dialysate may drain into a bag, a sink, or another location.

Because peritoneal dialysis uses the peritoneum, it is a risk factor for peritonitis. For example, peritoneal dialysis may inadvertently introduce bacteria to the abdomen. By some measures, there is about one incident of peritonitis per 24 to 48 patient-months. Peritonitis is a leading cause of mortality and treatment failure in peritoneal dialysis patients. Early detection proves difficult, and peritonitis is an invasive and rapidly developing bacteria, growing at rates as high as ten times per hour. Therefore, rapid diagnosis and treatment can be essential for therapeutic success.

Because of the risks associated with peritonitis, it may be important to monitor for symptoms and indicators of peritonitis in peritoneal dialysis patients. Indicators of peritonitis include, for example, increased white blood cell counts and differentiation. Spent dialysate (e.g., PD system effluent in a drain bag) may provide indicators of peritonitis. When peritonitis is sufficiently advanced, those indicators may even be visible to the naked eye. For example, spent dialysate may appear cloudy. Cloudiness may be assessed by various manual techniques, such as placing a newspaper under the drain bag and evaluating whether the letters are legible. When spent dialysate appears cloudy, patients are encouraged to perform another drain and bring the newly drained bag to a clinic for testing. In some cases, a patient may send a photo of the spent dialysate to a clinician, to obtain the clinician's subjective opinion based on its appearance. However, relying on such techniques means that peritonitis may not be detected until an infection is relatively advanced, particularly for patients with visual impairments, where it can be difficult for such patients to subjectively assess the “cloudiness” of spent dialysate. And by some estimates, over sixty percent of peritoneal dialysis patients have visual impairment.

As a reactive measure, patients may be placed on antibiotics before the clinician has a chance to properly test the spent dialysate. While the patient starts antibiotics, the clinician sends the spent dialysate to a lab, where it is inspected for white blood cell counts and bacteria. Depending on the lab results, the clinician may continue, discontinue, or change the patient's antibiotic treatment. Thus, traditional methods of monitoring for peritonitis are reactive, inefficient, and may delay appropriate medical treatment. In addition, traditional methods may result in unnecessary and/or partial antibiotic treatments in cases of false positives. For example, while the white cells in the effluent fluid exiting the patient during a PD cycle modifies the optical turbidity, the baseline color of the effluent fluid can vary in color and density.

There is a need for improved techniques for detecting peritonitis from a PD patient's effluent.

### SUMMARY

The present teachings include optical systems and methods for detection of peritonitis in early stages for patients undergoing a peritoneal dialysis (PD) treatment. An initial measurement of optical extinction of effluent fluid from a patient may be stored as a baseline value for the patient, e.g., to compensate for color, density, and similar optical factors related to the specific patient's physiology. Optical extinction through the effluent may be periodically measured over the course of a PD treatment, and extinction of signal relative to the baseline is analyzed, primarily for early indicators of potential peritonitis. The system may be externally connected to an effluent conduit, e.g., via a clip. A technique of ratioing an optical signal through effluent to a reference optical signal for measurements may provide high sensitivity while filtering out significant noise, enabling early detection of infection. Alerts of potential infection may be provided to the patient, caregiver, and/or medical team.

These and other features, aspects, and advantages of the present teachings will become better understood with reference to the following description, examples, and appended claims.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 is a schematic diagram of a system for early-stage detection of peritonitis in patients, in accordance with a representative embodiment.

FIG. 2 shows a system for early-stage detection of peritonitis in patients, in accordance with a representative embodiment.

FIG. 3 is a flow chart of a method to detect peritonitis, in accordance with a representative embodiment.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “of” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately” or the like, when accompanying a numerical value, are to be construed as indicating a deviation as would be appreciated by one of ordinary skill in the art to operate satisfactorily for an intended purpose. Similarly, words of approximation such as “about,” “approximately,” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” and the like, are words of convenience and are not to be construed as limiting terms unless specifically stated to the contrary.

In general, the devices, systems, and methods disclosed herein relate to use of an optical sensor for early detection of peritonitis during a peritoneal dialysis (PD) treatment, e.g., a Continuous Ambulatory Peritoneal Dialysis (CAPD) treatment and/or an Automated Peritoneal Dialysis (APD) treatment. To this end, the present teachings may include use of an initial measurement of optical extinction of effluent fluid from a patient (e.g., measured at the initiation of a treatment) that may be used as a baseline value for the patient, e.g., to compensate for color, density, and similar optical factors related to the specific patient's physiology at the time of measurement. Optical extinction through the effluent may then be periodically measured over the course of a dialysis treatment, where transmission relative to the baseline is analyzed, e.g., for early indicators of potential peritonitis.

Peritonitis can be deadly if left unchecked, but it can be difficult to detect early, which is problematic as the associated bacteria grows quickly—i.e., peritonitis is a very invasive and rapidly developing bacteria. Early detection can allow for rapid medical intervention prior to the peritonitis growing to serious levels. While the white blood cells in effluent fluid exiting a patient during a PD cycle can modify the effluent's optical turbidity, the baseline color of the effluent fluid can vary in color and density as described herein. Therefore, a non-calibrated optical sensor can mistake normal effluent fluid for turbid fluid and incorrectly indicate peritonitis.

The present teachings may thus involve use of an optical differential sensor that can be calibrated, prior to a PD treatment, e.g., relative to an empty cuvette in the effluent line or empty effluent tubing at a fixed location on the tubing. The reference signal intensity and the measurement intensity through the respective light sensors may be normalized mathematically to equal each other, which represents a condition with a transmission rate of 100%. Then, during the first drain cycle of effluent solution, a baseline percent transmission (defined as the ratio of light intensity received through the effluent solution divided by the reference signal sensor intensity) may be used as the “baseline” turbidity for the remainder of a PD treatment and effluent analysis. This allows for various colors and turbidity which may exist in various patients with underlying health conditions. During each cycle, or at other timeframes, a turbidity measurement may be compared to the baseline turbidity, where the difference data collected (both during a cycle and from cycle to cycle) throughout a PD treatment is analyzed for an increasing slope in turbidity indicating that the blocking of light is increasing. This analysis may occur, e.g., in real-time or near real-time during the treatment. A certain slope level or threshold of turbidity difference from the baseline can be established, which for example may activate an alert and/or send a notification that informs the patient and/or caregiver that peritonitis may be present and that the patient should seek further medical evaluation, or undergo some form of medical intervention. This approach may thus take into account various baseline colors and/or turbidity of diverse PD patients, while still allowing for a slope change detection technique to detect increases in white blood cells related to peritonitis bacteria growth.

Therefore, the present teachings may include techniques that are self-calibrating regardless of a patient's effluent fluid optical condition, which may be highly variable from patient to patient, or even highly variable from treatment to treatment for the same patient. Optical techniques such as those described herein may be useful in peritonitis detection because the presence of white blood cells due to peritonitis on-set will cause the effluent solution to increase in turbidity. And the change in this turbidity can be associated with the growth of peritonitis infection as additional white cells are produced by the body and included in the exiting effluent solution.

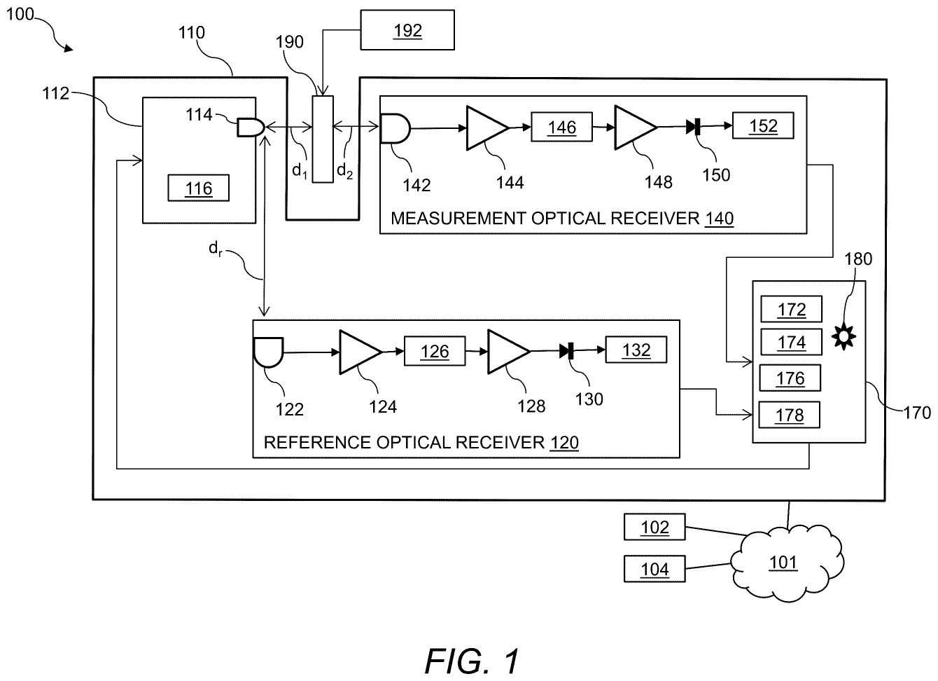

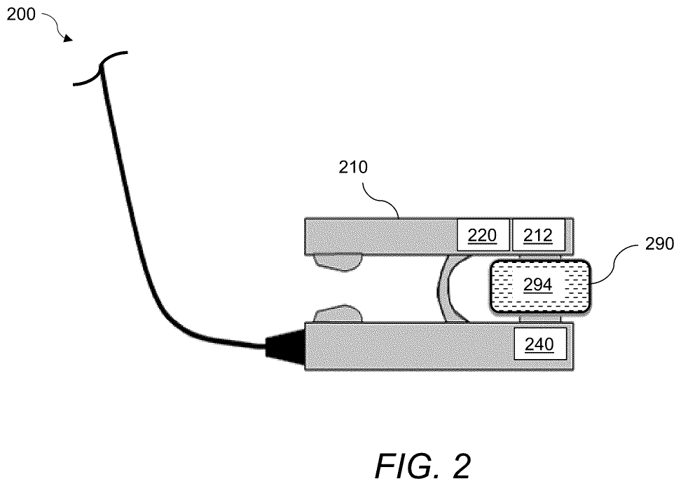

FIG. 1 shows a system for early-stage detection of peritonitis in patients, in accordance with a representative embodiment. The system 100 may include an optical differential PD effluent sensor 110 positioned about an effluent conduit 190 which may be connected (e.g., in fluid communication) with a PD system 192, which may include any as known in the art, such as an automated PD system, a CAPD system, or similar (for example, a system utilizing the Liberty® Select Cycler PD Machine by Fresenius Medical Care, North America, Waltham, Mass.). The optical differential PD effluent sensor 110 may generally include an optical transmitter 112, a reference optical receiver 120, a measurement optical receiver 140, and a controller 170. The optical differential PD effluent sensor 110 may be positioned about the effluent conduit 190 such that light transmitted by the optical transmitter 112 passes through the effluent conduit 190 and is incident on the measurement optical receiver 140 (refer also to discussion of FIG. 2 below). For example, the optical differential PD effluent sensor 110 may be positioned about the effluent conduit 190 such that the optical transmitter 112 and the measurement optical receiver 140 are located on opposing sides of a cuvette, an effluent tube, or another portion of the effluent conduit 190. The reference optical receiver 120 may be positioned to receive light transmitted by the optical transmitter 112 that is deflected away from, and does not pass through, the effluent conduit 190.

The optical transmitter 112 may include a light source 114 and a light source controller 116. It will be understood that the optical transmitter 112 may include any light source 114 which emits a substantially stable output over time (e.g., having a substantially stable output intensity, spectra, and the like). As an example, the light source 114 may include a light-emitting diode (LED) which may be driven with a substantially fixed current as set by the light source controller 116.

The light source 114 may include more than one LED, such as if additional optical power is desired at one or more of the optical receivers and/or to provide different spectral bands. The light source 114 may also, or instead, include a laser source, a fluorescent light source, an incandescent light source, and the like. In general, it may be desirable and beneficial for the light source 114 to emit light of a wavelength (or wavelength range) having low absorption by a PD solution. In some cases, light having a 600 nm wavelength may be considered to have low absorption by a PD solution. In an example case, the optical transmitter 112 may include a light source 114 that emits light of about a 660 nanometer wavelength (e.g., red color); this wavelength may be desirable for having low absorption by a PD solution. In another example case, the light source 114 may emit light of a wavelength having high absorption or scattering by white blood cells, (e.g., light of about a 580 nanometer wavelength). Such a system may have a relatively high sensitivity to changes in turbidity of the effluent fluid specifically due to an increase in white blood cells, and may therefore provide improved detection of early stage peritonitis (or other similar bacteria) compared to a method of detecting general changes in turbidity. In another example, light having a 600 nm wavelength may be considered to have high absorption or scattering by white blood cells. In some aspects, multiple wavelengths (e.g., multiple spectral bands) may be emitted by the optical transmitter 112.

Just as there may be some preferred wavelengths for the light source 114, there may also be some wavelength regions that may preferably be avoided. Examples include, in some cases, wavelengths around 800 nm (due to relatively high absorption and/or scattering by red blood cells) and/or around 1300 nm (due to relatively high absorption by water). Clearly, such preferences would depend on the specific conditions to be detected by the system, as made apparent in the discussion above where, in some instances, 800 nm light may be desired (e.g., to detect internal bleeding), and in other instances, 800 nm light may be proactively avoided.

The light source 114 and/or the optical transmitter 112 may include filters, lenses, polarizing elements, beam-shaping elements, and/or other optical elements as may be desired to modify characteristics of the emitted light, steer the optical path, and the like.

The light source controller 116 may be in electrical communication with the light source 114, and may provide a relatively stable current source to drive the light source 114. The light source controller 116 may also or instead include an amplitude modulator, a phase modulator, thermal controls, a signal generator, and the like; and/or the light source controller 116 may communicate with other system components to provide such features (e.g., the controller 170 may drive the light source controller 116, may provide input signals to the light source controller 116, and so on). The light source controller 116 may be operable to apply a modulation having a modulation frequency to the output of the light source 114.

The optical transmitter 112 may be arranged such that light emitted therefrom passes through the effluent conduit 190 and is incident on the measurement optical receiver 140. A portion of the emitted light may be deflected from this path, such as by a dome as described in U.S. Pat. Nos. 10,426,387 and 11,241,176, which are hereby incorporated by reference in their entirety. Also or instead, a portion of the emitted light may be deflected from this path by a beam-splitter or the like. The deflected light may be incident on the reference optical receiver 120.

The measurement optical receiver 140 may include a measurement photosensor 142, a transimpedance amplifier 144, a potentiometer 146, a bandpass filter 148, a rectifier 150, a match filter 152, a power source, or the like. While each of these are illustrated schematically as separate components, it is to be understood that some of these components may be packaged in a single device having multiple functions—by way of example, the measurement photosensor 142 and the potentiometer 146 may be packaged as a single device. The measurement optical receiver 140 may, similar to the optical transmitter 112, be temperature stable, mounted in a fixed distance and orientation from the optical transmitter 112, and be associated with a constant medium (or mediums) through which light will travel (e.g., through a cuvette or other section of effluent drain tubing through which the turbidity measurement is made).

The measurement photosensor 142, in general, may be sensitive to detecting light of the wavelength emitted by the optical transmitter 112. The type of measurement photosensor 142 may be, for example, a Silicon or Indium Gallium Arsenide photodiode or photodiode array, a photomultiplier, or the like. When light emitted by the optical transmitter 112 is incident on the measurement optical receiver 140 (e.g., after passing through the effluent conduit 190), the measurement photosensor 142 may generate a current proportional to the intensity of the detected light. The current from the measurement photosensor 142 may be passed to other components of the measurement optical receiver 140, such as the transimpedance amplifier 144 as shown.

The transimpedance amplifier 144 may receive a current from the measurement photosensor 142 and convert the current to a voltage. The transimpedance amplifier 144 may also perform other functions, such as amplification. The voltage output from the transimpedance amplifier 144 may be passed to other components of the measurement optical receiver 140, such as the potentiometer 146 as shown.

The potentiometer 146 may be, for example, a digital potentiometer or an analog potentiometer. The potentiometer 146 may be used, for example, to scale the voltage signal output by the transimpedance amplifier 144; in this manner, the dynamic range of the measurement optical receiver 140 may be adjusted. The voltage output from the potentiometer 146 may be passed to other components of the measurement optical receiver 140, such as the bandpass filter 148 as shown.

The bandpass filter 148 may be tuned to a frequency close to the modulation frequency applied by the light source controller 116. In this manner, the bandpass filter 148 may filter out unwanted signals due, for example, to stray light at the measurement photosensor 142, leakage current from the measurement photosensor 142, and so on. The output of the bandpass filter 148 may be rectified by a rectifier 150 and passed to the match filter 152 which may be synchronized to a modulation frequency applied by the light source controller 116 and may only pass signals having the same frequency (e.g., within a narrow frequency bandwidth).

The output signal from the measurement optical receiver 140 may be passed to the controller 170. Additional filtering, conditioning, and the like may be applied to the voltage signal from the measurement optical receiver 140, either within the measurement optical receiver 140 or within the controller 170.

The reference optical receiver 120 may include many of the same features described for the measurement optical receiver 140, such as a reference photosensor 122 that may be similar to the measurement photosensor 142, a transimpedance amplifier 124, a potentiometer 126, a bandpass filter 128, a rectifier 130, a match filter 132, or the like. The reference photosensor 122 may be the same, or similar to the measurement photosensor 142, and may generally be sensitive to detecting light of the wavelength emitted by the optical transmitter 112. The reference optical receiver 120 may be positioned such that a portion of the light emitted by the optical transmitter 112 (e.g., light deflected by a dome, or similar) is incident on the reference photosensor 122 without passing through the effluent conduit 190. The current output by the reference photosensor 122 may be passed to one or more components of the reference optical receiver 120 and filtered, conditioned, and/or processed by these components in the same or similar manner as described for the measurement optical receiver 140.

The controller 170 may receive signals from both the measurement optical receiver 140 and the reference optical receiver 120, and may process, analyze, transmit, and/or perform like operations on these signals. The controller 170 may include one or more analog to digital converters 172, a processor 174, a memory 176, a display 178, one or more indicators 180, and the like. The controller 170 may be in communication with one or more of the optical transmitter 112, the reference optical receiver 120, and the measurement optical receiver 140. The controller 170 may be configured to adjust one or more of an output intensity and an output wavelength of the optical transmitter 112, and may also or instead be configured to provide a reference signal for modulation of the light source 114 to either the light source controller 116 or the light source 114 itself. The controller 170 may be configured to synchronize the measurements of the reference optical receiver 120 and the measurement optical receiver 140 (e.g., using a time synchronized method to activate the reference photosensor 122 and the measurement photosensor 142 for a specific time interval, or at a specific time, e.g., relative to the output of the light source 114).

The controller 170 may thus include processing circuitry sufficient to drive the emissions of the optical transmitter 112, and/or synchronize the measurements of both the reference optical receiver 120 and measurement optical receiver 140. The controller 170, or another component of the system 100, may include processing circuitry to measure the trend in turbidity, and may also or instead be programmed to allow alerts, notifications, and the like when certain levels are achieved and/or rates of change are observed. The controller 170 may thus communicate to visual and/or digital indicators (e.g. data collection monitors and storage systems) to show the patient, assistant, clinician, physician, or others concerned with the patient's condition, whether peritonitis (or similar bacteria) is advancing the production of white cells at abnormal rates indicated by a drop in an optical signal.

Thus, the system 100 may include a controller 170, or otherwise be in communication with a controller 170, e.g., through a data network 101, which may be any as known in the art. The controller 170 may include, or otherwise be in communication with, a processor 174, a memory 176, a user device 102 such as a computing device, and so on, for controlling one or more of the components of the system 100, and/or for providing any of the analysis steps as described herein. Thus, in general, the controller 170 may be electronically coupled (e.g., wired or wirelessly) in a communicating relationship with one or more of the components of a system 100 for early-stage detection of peritonitis and the like. In general, the controller 170 may be operable to control the components of the system 100, and may include any combination of software and/or processing circuitry suitable for controlling the various components of the system 100 described herein including without limitation processors, microprocessors, microcontrollers, application-specific integrated circuits, programmable gate arrays, and any other digital and/or analog components, as well as combinations of the foregoing, along with inputs and outputs for transceiving control signals, power signals, sensor signals, and the like. In certain implementations, the controller 170 may include the processor 174 or other processing circuitry with sufficient computational power to provide related functions such as executing an operating system, providing a graphical user interface, setting and providing rules and instructions for operation of a component of the system 100, converting sensed information into instructions, notifications, and the like, and operating a web server or otherwise hosting remote operators and/or activity through one or more communications interfaces. In certain implementations, the controller 170 may include a printed circuit board, an Arduino controller or similar, a Raspberry Pi controller or the like, a prototyping board, or other computer related components.

The controller 170 may be a local controller disposed on the optical differential PD effluent sensor 110 or another component of the system 100, or a remote device otherwise in communication with the system 100 and its components—e.g., the controller 170 may be disposed on, or may include any components of, the remote computing resources 104 as described herein, and vice-versa. For example, one or more of the controller 170 and a user interface in communication with the controller 170 may be disposed on an external component (e.g., a user device 102 such as a smartphone with a display 178) in communication with the system 100 over a data network 101.

The optical differential PD effluent sensor 110—and/or another component in the system 100—may include one or more communications interfaces for, e.g., communication over the data network 101, or other communication between components of the devices or systems described herein. The communications interface may include, e.g., a Wi-Fi receiver and transmitter to allow the logic calculations to be performed on a separate computing device (e.g., the user device 102) and/or a remote computing resource 104. This may include connections to smartphone applications and the like. More generally, the communications interface may be suited such that any of the components of the system 100 can communicate with one another. Thus, the communications interface may be present on one or more of the components of the system 100. The communications interface may include, or be connected in a communicating relationship with, a network interface or the like. The communications interface may include any combination of hardware and software suitable for coupling the components of the system 100 to a remote device (e.g., a computing device such as a remote computer or the like) in a communicating relationship through a data network 101. By way of example and not limitation, this may include electronics for a wired or wireless Ethernet connection operating according to the IEEE 802.11 standard (or any variation thereof), or any other short or long range wireless networking components or the like. This may include hardware for short range data communications such as Bluetooth or an infrared transceiver, which may be used to couple into a local area network or the like that is in turn coupled to a data network 101 such as the internet. This may also or instead include hardware/software for a WiMAX connection or a cellular network connection (using, e.g., CDMA, GSM, LTE, or any other suitable protocol or combination of protocols). Additionally, the controller 170 may be configured to control participation by the components of the system 100 in any network to which the communications interface is connected, such as by autonomously connecting to the data network 101 to retrieve updates and the like.

The user devices 102 may include any devices within the system 100 operated by one or more users for practicing the techniques as contemplated herein. In general, a user device 102 may include a processor 174 and a memory 176, where the memory 176 stores computer-executable code embodied in a non-transitory computer-readable medium that, when executing by the processor 174, performs one or more steps of any method or technique described herein. Thus, although these components are shown on the controller 170 in FIG. 1, it will be understood that processing described herein may be accomplished using one or more other devices that also or instead include a processor 174 and a memory 176, such as the user device 102, the optical differential PD effluent sensor 110, and so on. In certain aspects, the user device 102 includes a dialysis machine and/or a component of a dialysis system. In this manner, in some aspects, the user is one or more of a dialysis patient, a dialysis technician, medical personnel such as a doctor or nurse, and so on. In other aspects, the user device 102 is a computing device associated with one or more of a medical professional, a data analyst, an emergency service provider, and the like. The user devices 102 may also or instead include any device for managing, monitoring, or otherwise interacting with tools, platforms, and devices included in the systems and techniques contemplated herein. The user devices 102 may be coupled to the data network 101, e.g., for interaction with one or more other participants in the system 100.

By way of example and not limitation, the user devices 102 may include one or more desktop computers, laptop computers, network computers, tablets, mobile devices, portable digital assistants, messaging devices, cellular phones, smart phones, portable media or entertainment devices, or any other computing devices that can participate in the system 100 as contemplated herein. The user devices 102 may also or instead include any form of mobile device, such as any wireless, battery-powered device, that might be used to interact with the system 100. It will also be appreciated that one of the user devices 102 may coordinate related functions (e.g., processing some or all data, storing data, etc.) as they are performed by another entity.

A user device 102, or another component of the system 100, may generally provide a user interface on a display 178. The user interface may be maintained by a locally executing application on one of the user devices 102 that receives data from, e.g., the remote computing resources 104 or other resources. In other embodiments, the user interface may be remotely served and presented on one of the user devices 102, such as where a remote computing resource 104 includes a web server that provides information through one or more web pages or the like that can be displayed (e.g., via the display 178) within a web browser or similar client executing on one of the user devices 102. The user interface may in general create a suitable visual presentation for user interaction on a display device of one of the user devices 102 or another component, and provide for receiving any suitable form of user input including, e.g., input from a keyboard, mouse, touchpad, touch screen, hand gesture, or other user input device(s).

The remote computing resources 104 may include, or otherwise be in communication with, a processor 174 and a memory 176, where the memory 176 stores code executable by the processor 174 to perform various techniques of the present teachings. More specifically, a remote computing resource 104 may be coupled to the data network 101 and accessible to a component of the system 100 through the data network 101, e.g., where the remote computing resource 104 includes a processor 174 and a memory 176.

The processor 174 may include an onboard processor for the controller 170, the optical differential PD effluent sensor 110, and/or another component of the system 100. The processor 174 may also or instead be disposed on a separate computing device that is connected to the system 100 or one or more of its components through a data network 101, e.g., using a communications interface, which may include a Wi-Fi transmitter and receiver. The processor 174 may perform calculations and/or other analyses such as any as described herein. The processor 174 may be any as described herein or otherwise known in the art. The processor 174 may be included on the controller 170, or it may be separate from the controller 170, e.g., it may be included on a user device 102 in communication with the controller 170 or another component of the system 100, a remote computing resource 104, and so on. In an implementation, the processor 174 is included on, or is in communication with, a server that hosts an application for operating and controlling the system 100.

The memory 176 may be any as described herein or otherwise known in the art. The memory 176 may contain computer code and may store data such as sequences of operation for one or more of the components of the system 100 (e.g., the optical differential PD effluent sensor 110), sequences or content for notifications and alerts, historical data (e.g., previous inputs, measurements, and calculations), and so on. The memory 176 may also or instead contain computer executable code stored thereon that provides instructions for the processor 174 for implementation. The memory 176 may include a non-transitory computer readable medium.

The remote computing resources 104 may also or instead include data storage, a network interface (and/or other communications interface(s)), and/or other processing circuitry. In the following description, where the functions or configuration of a remote computing resource 104 are described, this is intended to include corresponding functions or configuration (e.g., by programming) of a processor 174 of the remote computing resource 104, or in communication with the remote computing resource 104. In general, the remote computing resources 104 (or processors 174 thereof or in communication therewith) may perform a variety of processing tasks related to detecting peritonitis in early stages in PD patients and the like as discussed herein. For example, the remote computing resources 104 may manage information received from a component of the system 100, and provide related supporting functions, processing one or more data sets, communicating with other resources, storing data, and the like. The remote computing resources 104 may also or instead include backend algorithms that react to actions performed by a user or component of the system 100. The remote computing resources 104 may also or instead include a web server or similar front end that facilitates web-based access by the user devices 102 to the capabilities of the remote computing resource 104 or other components of the system 100. A remote computing resource 104 may also or instead communicate with other resources in order to obtain information for providing to a user through a user interface on the user device 102. A remote computing resource 104 may also or instead maintain, or otherwise be in communication with, a database of content such as data related to a plurality of dialysis patients or the like, values for calculations and/or results thereof, threshold settings, and the like. The database may thus be used to store any raw and/or processed data as described herein, e.g., for use by another component in the system 100.

It will thus be understood that the participants in the system 100 may include any hardware or software to perform various functions as described herein.

The effluent conduit 190 may generally be configured to transport effluent fluid exiting the patient during a PD treatment, e.g., the effluent conduit 190 may be part of a PD treatment's drainage system. The effluent conduit 190 may thus include tubing, valves, filters, and the like. In an aspect, the system 100 may operate in cooperation with the effluent conduit 190, while in other aspects, the system 100 may include the effluent conduit 190. The optical differential PD effluent sensor 110 may be positioned about the effluent conduit 190, preferably in a region that exhibits relatively low optical transmission losses when the effluent conduit 190 is free of effluent. This region may, for example, include a portion of an effluent drain tube or a cuvette or rigid chamber that may be connected intermediate the effluent drain tube and may be selected for having specific optical properties.

The position of the optical transmitter 112 relative to the effluent conduit 190 (e.g., the distance d₁, the angle between the optical transmitter 112 and the effluent conduit 190, and the like) may be substantially constant, for example, over the course of a PD treatment. The medium (e.g., air, a cuvette wall, a tubing wall, or the like) through which the light travels from the optical transmitter 112 to the effluent conduit 190 over the distance d₁, may also be constant, and may have a composite optical extinction coefficient, e₁, for the light emitted by the optical transmitter 112 (e.g., e₁ may be specific to the wavelength of light emitted by the optical transmitter 112). Similarly, the position of the measurement optical receiver 140 relative to the effluent conduit 190 (e.g., the distance d₂, the angle between the measurement optical receiver 140 and the effluent conduit 190, and the like) may be substantially constant. The medium within the distance d₂ may also be substantially constant, and may have a composite optical extinction coefficient, e₂, relative to the light transmitted by the optical transmitter 112. In addition, the position of the optical transmitter 112 relative to the reference optical receiver 120 (e.g., the distance dᵣ, the angle therebetween, and the like) may be substantially constant. The medium within the distance dᵣ may also be substantially constant, and may have a composite optical extinction coefficient, eᵣ, relative to the light transmitted by the optical transmitter 112.

FIG. 2 shows a system 200 for early-stage detection of peritonitis in patients, in accordance with a representative embodiment. The system 200 may have many features in common with the system 100 of FIG. 1, such as an optical differential PD effluent sensor 210 positioned about an effluent conduit 290. Thus, it will be understood that the system 200 of FIG. 2 may share any of the above-mentioned features of the system 100 of FIG. 1, and vice-versa.

The optical differential PD effluent sensor 210 may have a form similar to the CRIT-LINE® monitoring system produced by Fresenius Medical Care of Waltham, Mass. The optical differential PD effluent sensor 210 may, for example, be clipped onto the effluent conduit 290 thereby maintaining a substantially constant position of the optical differential PD effluent sensor 210 relative to the effluent conduit 290 over the duration of a PD treatment. The optical differential PD effluent sensor 210 may include an optical transmitter 212, a reference optical receiver 220, and a measurement optical receiver 240, e.g., in an arrangement substantially as described with reference to the optical differential PD effluent sensor 110 of FIG. 1. The effluent conduit 290 may include a medium 294, and may be substantially filled with the medium. The medium 294 may be effluent from a PD system, such as when the optical differential PD effluent sensor 210 is positioned for use for monitoring. The medium 294 may instead be another substance, such as air or water, for example when establishing a baseline transmission for the optical differential PD effluent sensor 210.

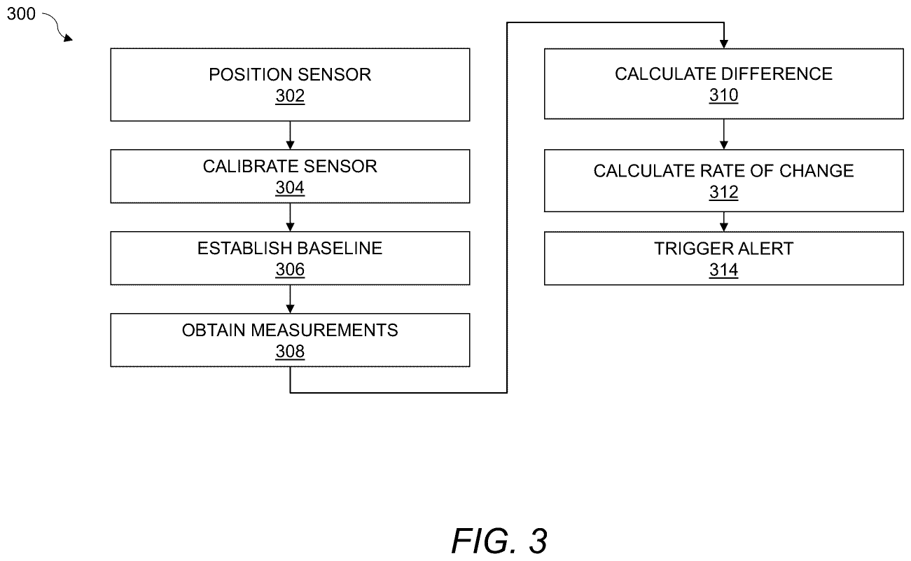

FIG. 3 is a flow chart of a method 300 to detect peritonitis in early stages in patients undergoing a PD treatment, in accordance with a representative embodiment. The method 300 may be implemented using any of the devices or systems described herein, such as the system 100 of FIG. 1 and/or the system 200 of FIG. 2.

As shown in step 302, the method 300 may include positioning a sensor, e.g., an optical differential PD effluent sensor (e.g., the optical differential PD effluent sensor 110), about an effluent conduit of a PD treatment system (e.g., the effluent conduit 190). Positioning the sensor may include clipping the sensor onto a portion of the effluent conduit, as shown and described in FIG. 2. This step 302 may also include connecting a cuvette to the effluent conduit.

As shown in step 304, the method 300 may include calibrating a sensor (e.g., the optical differential PD effluent sensor 110). Calibrating the sensor may be performed prior to initiating the PD treatment, and may be specific to parameters of the PD treatment system and/or session. In other words, this calibration step may be distinct from a “factory” calibration of the system (that may, e.g., be performed to set operating frequencies, sensor gains, and the like), although these or similar operations may also be performed as part of this step 304. A signal intensity, IMO, may be measured by the measurement optical receiver (e.g., the measurement optical receiver 140) while the effluent conduit is empty of effluent (e.g., the medium in the effluent conduit may be air, water, or another medium having known optical properties). Simultaneously, a signal intensity, IRO, may be measured by the reference optical receiver (e.g., the reference optical receiver 120). A ratio of the signal intensities may be calculated to obtain a normalization constant, S, as shown in Eq. 1 below.

$$S = {I}_{M 0} / {I}_{R 0} \qquad Eq . 1$$

The signal intensities at each receiver are described by Beer's Law:

$${I}_{M O} = {I}_{0} * {e}^{- e 1 d {1}_{*}} {e}^{- e s d s} * {e}^{- e 2 d 2} \qquad Eq . 2$$

$${I}_{R O} = {I}_{0} * {e}^{- erdr} \qquad Eq . 3$$

In the equations above, I₀ is the intensity of light emitted by the optical transmitter; optical extinction coefficients e₁, e₂, and eᵣ, are described with reference to FIG. 1, and distances coefficients d₁, d₂, and dᵣ, are as shown in FIG. 1. The optical extinction coefficient of the sample, eₛ, (e.g., the effluent conduit 190 and medium contained therein) is taken over the sample pathlength, dₛ. When the sample chamber is empty, the extinction coefficient eₛ is approximately zero, yielding Eq. 4.

$${e}^{- e s d s} = 1 \qquad Eq . 4$$

By substituting Eqs. 2-4 into Eq. 1, the constant S is shown represented below in Eq. 5.

$$S = {e}^{- e 1 d 1} * {e}^{- e 2 d 2} * {e}^{+ erdr} \qquad Eq . 5$$

The calculation of Eq. 1, and calculations of other steps of the method 300, may be performed, for example, by a processor (e.g., the processor 174). The processor may be configured to receive the signal intensities from the optical receivers (e.g., from the digital converters 172 of the controller 170).

As shown in step 306, the method 300 may include establishing a baseline percent transmission, %T_{B}. The baseline percent transmission provides a measurement that may be used for comparison purposes over the course of a PD treatment, and may be used to compensate for differences in effluent turbidity, color, and the like between patients and/or between treatment sessions for the same patient. As such, the baseline percent transmission may be associated with a specific treatment session for a specific patient. Establishing the baseline percent transmission may occur, for example, during a first drain cycle of the PD treatment. The baseline transmission may be calculated as shown in Eq. 6 below, where I_{M,B} is a signal intensity measured through effluent solution (e.g., through the effluent conduit 190 containing effluent solution) by the measurement optical receiver; I_{R,B} is a signal intensity measured by the reference optical receiver, e.g., simultaneously to the measurement of I_{M,B}; and S is calculated in step 304.

$$\% {T}_{B} = {I}_{M , B} / ( {I}_{R , B} * S ) \qquad Eq . 6$$

As shown in step 308, the method 300 may include obtaining a plurality of treatment percent transmission measurements, %T_{N}, (where N is an integer value indicating one of the plurality of treatment percent transmission measurements, with N=1 indicating the first measurement and N representing the most recent measurement). The treatment percent transmission measurements may be separated by a predetermined interval (e.g., a time interval) during the PD treatment. For example, for a PD treatment that may include from 4-6 cycles per session, the predetermined interval may occur at least once per cycle of the PD treatment. In another example, the predetermined interval may be a fixed time interval, such as at least once per day, at least one per hour, and so on. The treatment percent transmission may be calculated as shown in Eq. 7, where I_{M,N} is a signal intensity through the effluent solution (e.g., through the effluent conduit 190 containing effluent solution) by the measurement optical receiver at measurement N; I_{R,N} is a signal intensity measured by the reference optical receiver at measurement N, e.g., simultaneously to the measurement of I_{M,N}; and S is calculated in step 304.

$$\% {T}_{N} = {I}_{M , N} / ( {I}_{R , N} * S ) \qquad Eq . 7$$

As shown in step 310, the method 300 may include calculating a percent transmission difference, Δ%T. The percent transmission difference may be calculated as the difference between the treatment percent transmission measurement, %T_{N}, and the baseline percent transmission, %T_{B}, as shown in Eq. 8 below.

$$Δ \% T = \% {T}_{B} - \% {T}_{N} \qquad Eq . 8$$

The percent transmission difference may be calculated for each of the plurality of treatment percent transmission measurements (e.g., for N=1, 2, . . . (N−1), N). Depending on the specific parameters of the system, such as operating wavelength, the percent transmission difference may correlate to one or more of a turbidity of an effluent solution and a concentration of white blood cells. The percent transmission difference may, in some cases, be used to trigger an alert as described in step 314. The percent transmission difference may be stored, e.g., in a memory of a controller or the like, and/or communicated to a database by a controller or the like.

As shown in step 312, the method 300 may include calculating a rate of change, ROC, between at least two of the percent transmission differences (e.g., as obtained in step 310). The rate of change may be calculated, for example, between two consecutive measurements (e.g., from measurement (N−1) to measurement N); between all measurements of a PD cycle; between one or more measurements of consecutive PD cycles; and the like. An example of the calculation of ROC over two consecutive measurements is given in Eq. 9 below.

$$R O C = \frac{\% {T}_{N} - \% {T}_{( N - 1 )}}{( N - ( N - 1 ) )} \qquad Eq . 9$$

The ROC may be analyzed, for example, to detect a decreasing percent transmission indicating an increase in turbidity of the effluent. The ROC may be displayed, e.g., graphically, such as on a display of a controller or the like. The ROC may be stored, e.g., in a memory of a controller or the like, and/or communicated to a database by a controller or the like.

As shown in step 314, the method 300 may include triggering an alert. An alert may be triggered, for example, when the rate of change calculated in step 312 is above a threshold rate, e.g., where the threshold rate is selected to be indicative of peritonitis. The alert may be provided to, e.g., the patient, a caregiver, a medical professional, or similar, and may indicate the possible presence of peritonitis and/or may recommend a medical evaluation. The alert may include one or more of a visual indicator (e.g., activating one or more indicators of a controller or the like), an audible alert, and a communication a computing device, such as a device of the patient, medical personnel, caregiver, and the like.

It will be understood that the method 300 described above, and/or other techniques and methods described herein, may be performed at least in part by a computer program product and/or a component of a PD treatment system. For example, in an aspect, a computer program product for detecting peritonitis in early stages in patients undergoing a PD treatment may include computer executable code embodied in a non-transitory computer readable medium that, when executing on one or more computing devices, performs the steps of: calibrating an optical differential PD effluent sensor, the sensor positioned about an effluent conduit of a PD treatment system, the sensor including an optical transmitter, a reference optical receiver, and a measurement optical receiver, such that light transmitted by the optical transmitter passes through the effluent conduit and is incident on the measurement optical receiver, the calibration including, prior to initiating a PD treatment, ratioing a signal intensity, Iₘ₀, from the measurement optical receiver while the effluent conduit is empty, to a signal intensity, Iᵣ₀, from the reference optical receiver, to obtain a normalization constant, S; establishing a baseline percent transmission, during a first drain cycle of the PD treatment, as a ratio of a signal intensity, Iₘ, measured through effluent solution to a value of the signal intensity from the reference optical receiver multiplied by the normalization constant, Iᵣ×S; obtaining a plurality of treatment percent transmission measurements separated by a predetermined interval during the PD treatment; calculating, for each of the plurality of treatment percent transmission measurements, a percent transmission difference between a treatment percent transmission measurement and the baseline percent transmission; calculating a rate of change between at least two percent transmission differences; and triggering an alert when the rate of change is above a threshold rate, the threshold rate selected to be indicative of peritonitis.

The above systems, devices, methods, processes, and the like may be realized in hardware, software, or any combination of these suitable for a particular application. The hardware may include a general-purpose computer and/or dedicated computing device. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. In another aspect, the methods may be embodied in systems that perform the steps thereof, and may be distributed across devices in a number of ways. At the same time, processing may be distributed across devices such as the various systems described above, or all of the functionalities may be integrated into a dedicated, standalone device or other hardware. In another aspect, means for performing the steps associated with the processes described above may include any of the hardware and/or software described above. All such permutations and combinations are intended to fall within the scope of the present disclosure.

Embodiments disclosed herein may include computer program products comprising computer-executable code or computer-usable code that, when executing on one or more computing devices, performs any and/or all of the steps thereof. The code may be stored in a non-transitory fashion in a computer memory, which may be a memory from which the program executes (such as random-access memory associated with a processor), or a storage device such as a disk drive, flash memory or any other optical, electromagnetic, magnetic, infrared, or other device or combination of devices. In another aspect, any of the systems and methods described above may be embodied in any suitable transmission or propagation medium carrying computer-executable code and/or any inputs or outputs from same.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings.

Unless the context clearly requires otherwise, throughout the description, the words “comprise,” “comprising,” “include,” “including,” and the like are to be construed in an inclusive sense as opposed to an exclusive or exhaustive sense; that is to say, in a sense of “including, but not limited to.” Additionally, the words “herein,” “hereunder,” “above,” “below,” and words of similar import refer to this application as a whole and not to any particular portions of this application.

It will be appreciated that the devices, systems, and methods described above are set forth by way of example and not of limitation. For example, regarding the methods provided above, absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to include any suitable method of causing such method steps to be performed, consistent with the patentability of the following claims, unless a different meaning is expressly provided or otherwise clear from the context. So, for example performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

While particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A method of detecting peritonitis in early stages in patients undergoing a peritoneal dialysis (PD) treatment, the method comprising:
positioning an optical differential PD effluent sensor about an effluent conduit of a PD treatment system, the sensor including an optical transmitter, a reference optical receiver, and a measurement optical receiver, such that light transmitted by the optical transmitter passes through the effluent conduit and is incident on the measurement optical receiver;
calibrating the sensor, prior to initiating a PD treatment, by ratioing a signal intensity, Iₘ₀, from the measurement optical receiver while the effluent conduit is empty, to a signal intensity, Iᵣ₀, from the reference optical receiver, to obtain a normalization constant, S;
establishing a baseline percent transmission, during a first drain cycle of the PD treatment, as a ratio of a signal intensity, Iₘ, measured through effluent solution to a value of the signal intensity from the reference optical receiver multiplied by the normalization constant, Iᵣ×S;
obtaining a plurality of treatment percent transmission measurements separated by a predetermined interval during the PD treatment;
calculating, for each of the plurality of treatment percent transmission measurements, a percent transmission difference between a treatment percent transmission measurement and the baseline percent transmission;
calculating a rate of change between at least two percent transmission differences; and
triggering an alert when the rate of change is above a threshold rate, the threshold rate selected to be indicative of peritonitis.

2. The method of claim 1, wherein the percent transmission difference correlates to one or more of: a turbidity of effluent solution and a concentration of white blood cells.

3. The method of claim 1, wherein the predetermined interval occurs at least once per cycle of the PD treatment.

4. The method of claim 1, wherein the predetermined interval occurs at least once per day.

5. The method of claim 1, wherein the baseline percent transmission is associated with a specific treatment session for a specific patient.

6. The method of claim 1, wherein positioning the sensor about the effluent conduit includes positioning the optical transmitter and the measurement optical receiver on opposing sides of a cuvette.

7. The method of claim 1, wherein positioning the sensor about the effluent conduit includes positioning the optical transmitter and the measurement optical receiver on opposing sides of a portion of tubing.

8. The method of claim 1, wherein the optical transmitter emits light of a wavelength having low absorption by a peritoneal dialysis solution.

9. The method of claim 8, wherein the optical transmitter emits light of about a 660 nanometer wavelength.

10. The method of claim 1, wherein the optical transmitter emits light of a wavelength having high absorption or scattering by white blood cells.

11. The method of claim 10, wherein the optical transmitter emits light of about a 580 nanometer wavelength.

12. The method of claim 1, wherein the sensor includes a controller in communication with one or more of the optical transmitter, the reference optical receiver, and the measurement optical receiver.

13. The method of claim 12, wherein the controller is configured to synchronize measurements of the reference optical receiver and the measurement optical receiver.

14. The method of claim 12, wherein the controller is configured to adjust one or more of an output intensity and an output wavelength of the optical transmitter.

15. The method of claim 1, wherein the alert includes one or more of a visual indicator, an audible alert, and a communication transmitted to a computing device.

16. The method of claim 1, wherein the effluent conduit is connected to an automated peritoneal dialysis system.

17. The method of claim 1, wherein the effluent conduit is connected to a continuous ambulatory peritoneal dialysis system.

18. A computer program product for detecting peritonitis in early stages in patients undergoing a peritoneal dialysis (PD) treatment, the computer program product comprising computer executable code embodied in a non-transitory computer readable medium that, when executing on one or more computing devices, performs the steps of:
calibrating an optical differential PD effluent sensor, the sensor positioned about an effluent conduit of a PD treatment system, the sensor including an optical transmitter, a reference optical receiver, and a measurement optical receiver, such that light transmitted by the optical transmitter passes through the effluent conduit and is incident on the measurement optical receiver, the calibration comprising: prior to initiating a PD treatment, ratioing a signal intensity, Iₘ₀, from the measurement optical receiver while the effluent conduit is empty, to a signal intensity, Iᵣ₀, from the reference optical receiver, to obtain a normalization constant, S;
establishing a baseline percent transmission, during a first drain cycle of the PD treatment, as a ratio of a signal intensity, Iₘ, measured through effluent solution to a value of the signal intensity from the reference optical receiver multiplied by the normalization constant, Iᵣ×S;
obtaining a plurality of treatment percent transmission measurements separated by a predetermined interval during the PD treatment;
calculating, for each of the plurality of treatment percent transmission measurements, a percent transmission difference between a treatment percent transmission measurement and the baseline percent transmission;
calculating a rate of change between at least two percent transmission differences; and
triggering an alert when the rate of change is above a threshold rate, the threshold rate selected to be indicative of peritonitis.

19. A peritoneal dialysis (PD) system for early-stage detection of peritonitis in patients, the system comprising:
an optical differential PD effluent sensor positioned about an effluent conduit, the sensor including an optical transmitter, a reference optical receiver, and a measurement optical receiver, the sensor positioned such that light transmitted by the optical transmitter passes through the effluent conduit and is incident on the measurement optical receiver; and
a memory and one or more processors, the memory storing code executable by the one or more processors to:
receive a signal intensity, Iₘ₀, from the measurement optical receiver while the effluent conduit is empty and a signal intensity, Iᵣ₀, from the reference optical receiver;
calculate a normalization constant, S, as a ratio of Iₘ₀ to Iᵣ₀;
establish a baseline percent transmission, during a first drain cycle of a PD treatment, as a ratio of the signal intensity, Iₘ, measured through effluent solution to a value of the signal intensity from the reference optical receiver multiplied by the normalization constant, Iᵣ×S;
obtain a plurality of treatment percent transmission measurements separated by a predetermined interval during the PD treatment;
calculate, for each of the treatment percent transmission measurements, a percent transmission difference between the treatment percent transmission measurement and the baseline percent transmission;
calculate a rate of change between at least two of the percent transmission differences; and
trigger an alert when the rate of change is above a threshold rate, the threshold rate selected to be indicative of peritonitis.

20. The system of claim 19, further comprising an effluent conduit configured to transport effluent fluid exiting the patient.
